(19) United States
(12) Patent Application Publication
(10) Pub. No.: US 2006/0004300 A1
(43) Pub. Date: Jan. 5, 2006

(54) Multifrequency bioimpedance determination

(76) Inventor: James Kennedy, St. Lucia (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/134,824

(22) Filed: May 20, 2005

Related U.S. Application Data
(63) Continuation of application No. PCT/AU03/01566, filed on Nov. 21, 2003.
(60) Provisional application No. 60/429,047, filed on Nov. 22, 2002.

(30) Foreign Application Priority Data
Nov. 22, 2002 (AU) 2002952840

(51) Int. Cl. A61B 5/05

(52) U.S. Cl. 600/547

(57) ABSTRACT

A method of determining the impedance of a subject is provided. This method involves applying an electrical signal representing a range of superimposed frequencies, and then determining the current flow through and voltage across the subject for a number of the frequencies within the range. The impedance of the subject is then determined at each of the number of frequencies. An apparatus and a processing system configured for use in impedance determination are also provided.

### RELATED APPLICATIONS

This application is a continuation under 35 U.S.C. § 120 of the International Patent Application No. PCT/AU03/01566, filed on Nov. 21, 2003, and published in English on Jun. 10, 2004, which claims the benefit of Australian Patent Application No. 2002952840, filed on Nov. 22, 2002 and U.S. Provisional Application 60/429,047, filed on Nov. 22, 2002, each of which is incorporated by reference.

### BACKGROUND OF THE INVENTION

The present invention relates to a method and apparatus for determining the impedance of a subject, and in particular to determining the biological impedance of a biological subject.

### DESCRIPTION OF THE RELATED TECHNOLOGY

The reference to any technology in this specification is not, and should not be taken as, an acknowledgement or any form of suggestion that the technology forms part of the common general knowledge.

Correlations between whole-body impedance measurements and various body characteristics, such as total body water (TBW) and fat-free mass (FFM), are experimentally well established. As a consequence, bioelectrical impedance analysis (BIA) is widely used in human nutrition and clinical research.

It is generally accepted that BIA provides a reliable estimate of total body water under most conditions and in the National Institutes of Health Technology Assessment Statement entitled “Bioelectrical Impedance Analysis in Body Composition Measurement, Dec. 12-14, 1994” it was noted that BIA can be a useful technique for body composition analysis in healthy individuals and in those with a number of chronic conditions such as mild-to-moderate obesity, diabetes mellitus, and other medical conditions in which major disturbances of water distribution are not prominent. In addition, BIA is fast, inexpensive, and does not require extensive operator training or cross-validation.

BIA measures the impedance or opposition to the flow of an electric current through the body fluids contained mainly in the lean and fat tissue. Impedance is low in lean tissue, where intracellular fluid and electrolytes are primarily contained, but high in fat tissue. Impedance is thus proportional to TBW.

Currently, in practice, a small constant current, typically 800 μA at a fixed frequency, usually 50 kHz, is passed between electrodes spanning the body and the voltage drop between electrodes provides a measure of impedance. Prediction equations, previously generated by correlating impedance measures against an independent estimate of TBW, may be used subsequently to convert a measured impedance to a corresponding estimate of TBW. Lean body mass is then calculated from this estimate using an assumed hydration fraction for lean tissue. Fat mass is calculated as the difference between body weight and lean body mass.

The impedance of a biological tissue comprises two components, resistance and reactance. The conductive characteristics of body fluids provide the resistive component, whereas the cell membranes, acting as imperfect capacitors, contribute a frequency-dependent reactive component. Impedance measurements made over a range of low to high (1 MHz) frequencies therefore allow development of prediction equations relating impedance measures at low frequencies to extracellular fluid volume and at high frequencies to total body fluid volume. This is often referred to as multi-frequency bioelectrical impedance analysis.

Recent applications of BIA increasingly use multi-frequency measurements, or a frequency spectrum, to evaluate differences in body composition caused by clinical and nutritional status. While the National Institutes of Health Technology Assessment Statement did not support the use of BIA under conditions that alter the normal relationship between the extracellular (ECW) and intracellular water (ICW) compartments, recent studies indicate that the only model that accurately predicted change in ECW, ICW, and TBW is the zero-infinity kHz parallel multiple frequency model, often referred to as a Cole-Cole plot (example, refer Gudivaka, R., D. A. Schoeller, R. F. Kushner, and M. J. G. Bolt. Single- and multi-frequency models for bioelectrical impedance analysis of body water compartments. J. Appl. Physiol. 87(3): 1087-1096, 1999).

Currently techniques for implementing multi-frequency analysis involve applying a number of signals to the subject in turn, with each signal having a respective frequency. The resulting impedance at each frequency is then determined separately, allowing the dependence of impedance on frequency to be determined. An example of apparatus suitable for performing impedance determination using this technique is shown in U.S. Pat. No. 5,280,429.

In this case, once the impedance at each frequency has been obtained, and the results are plotted as a graph of resistance versus frequency, reactance versus frequency, of resistance versus reactance (the zero-infinity kHz parallel multiple frequency plot, or Cole-Cole plot, referred to above).

However, this technique suffers from a number of drawbacks. In particular, it is necessary to generate a large number of data points for accurate plots to be made. Furthermore, as each respective frequency signal must be applied to the subject in turn, this procedure can take a long time, and in particular, can take as long as half-an-hour.

### SUMMARY OF CERTAIN INVENTIVE ASPECTS

In one embodiment, a method of determining the impedance of a subject is provided, the method including:
a) Applying an electrical signal representing a range of superimposed frequencies;
b) Determining for a number of frequencies within the range:
   i) The current flow through the subject; and,
   ii) The voltage across the subject; and,
c) Determining the impedance of the subject at each of the number of frequencies.

In further embodiments, the method includes:
a) Generating component signals, each component signal having a respective one of the number of frequencies; and,
b) Superimposing the component signals to generate the electrical signal.

In alternate embodiments the electrical signal can be formed from white noise. In one such embodiment, the method includes:
a) Generating the white noise using a Linear Feedback Shift Register (LFSR) circuit to produce a pseudo-random digital sequence; and,
b) Converting the pseudo-random digital sequence to an analog signal using a digital to analog (D/A) converter; and,
c) Applying the analog signal to the subject.

In further embodiments, the method of determining the current flow includes:
a) Sampling the current of the electrical signal applied to the subject; and,
b) Converting the current signal to a digitized current signal.

In further embodiments, the method of determining the voltage generally includes:
a) Obtaining a signal representing the voltage generated across the subject;
b) Converting the voltage signal to a digitized voltage signal.

The method can include digitizing the current and voltage signals by sampling the signals at a predetermined rate. Furthermore, the method can include digitizing the current and voltage signals by sampling the signals with a predetermined sample length. It will be appreciated that a range of values may be used for the predetermined rate, such as several MHz, with the sample length typically being up to a thousand or so sample points or more, depending on the implementation.

In certain embodiments, the method includes converting each of the digitized voltage and current signals into the frequency domain. This conversion may be performed using a Fast Fourier Transform (FFT).

The method can include using a processing system to:
a) Receive the converted voltage and current signals; and,
b) Determine the impedance of the subject at each of the number of frequencies.

The processing system can be further adapted to determine the variation in the impedance with the frequency of the applied signal.

In certain embodiments, the method further includes generating a graphical representation of the variation in the impedance with the frequency of the applied signal.

In an alternative embodiment, an apparatus for determining the impedance of a subject is provided, the apparatus including:
a) A signal generator for applying an electrical signal representing a range of superimposed frequencies;
b) A voltage detector for determining the voltage across the subject at a number of frequencies within the range;
c) A current detector for determining the current flow through the subject at a number of frequencies within the range; and,
d) A processing system for determining the impedance of the subject at each of the number of frequencies.

In a further embodiment, the signal generator can be adapted to:
a) Generate component signals, each component signal having a respective one of the number of frequencies; and,
b) Superimpose the component signals to generate the electrical signal.

Alternatively, the electrical signal can be formed from white noise, in which case the signal generator typically includes:
a) A shift register circuit to produce a pseudo-random digital sequence; and,
b) A D/A converter for converting the pseudo-random digital sequence to an analog signal.

In one embodiment, the shift register circuit includes:
a) A shift register having an output coupled to the D/A converter; and,
b) An exclusive OR (XOR) gate adapted to:
   i) Receive inputs from a number of predetermined locations in the first register;
   ii) Logically combine the inputs to generate an XOR output; and,
   iii) Provide the XOR output to an input of the shift register;

The signal generator can include a second shift register, the second shift register being adapted to couple an output of the first shift register to an input of the D/A converter.

In certain embodiments the current detector includes:
a) A current sampler coupled to the signal generator for sampling the current flowing through the subject; and,
b) A current analog to digital (A/D) converter for converting the sampled current to a digitized current signal.

In certain embodiments, the voltage detector includes a voltage A/D converter coupled to the subject via a respective set of electrodes, the voltage A/D converter being adapted to generate a digitized voltage signal.

The current and voltage A/D converters may be adapted to digitize the current and voltage signals by sampling the signals at a predetermined rate, and/or by sampling the signals with a predetermined sample length. As mentioned above however, alternative sample rates and lengths may be used.

In certain embodiments, the processing system is adapted to convert each of the digitized voltage and current signals into the frequency domain. This may be performed using a FFT.

The processing system may include processing electronics for performing the conversion.

The processing system can be adapted to:
a) Receive the converted voltage and current signals; and,
b) Determine the impedance of the subject at each of the number of frequencies.

The processing system can be further adapted to determine the variation in the impedance with the frequency of the applied signal.

The processing system can be further adapted to generating a graphical representation of the variation in the impedance with the frequency of the applied signal.

In yet another embodiment a processing system for use in apparatus for determining the impedance of a subject is provided, the processing system being adapted to:
a) Receive a digitized current signal representing the current flow through the subject at a number of frequencies for an applied electrical signal representing a range of superimposed frequencies;
b) Receive a digitized voltage signal representing the voltage across the subject at a number of frequencies within the range;
c) Convert each digitized signal into the frequency domain; and,
d) Determine the impedance of the subject at each of the number of frequencies.

The conversion can be performed using a FFT.

The processing system can include processing electronics for performing the conversion.

In certain embodiments, the processing system includes a processor for determining the impedance.

The processor can be further adapted to determine the variation in the impedance with the frequency of the applied signal.

In certain embodiments, the processing system includes a display, the processor being adapted to generating a graphical representation of the variation in the impedance with the frequency of the applied signal.

In yet another embodiment, a computer program product for determining the impedance of a subject is provided, the computer program product including computer executable code which when executed by a suitable processing system causes the processing system to operate as the processing system of the third broad form of the invention.

### BRIEF DESCRIPTION OF THE DRAWINGS

An embodiment of the present invention will now be described with reference to the accompanying drawings, in which:—

FIG. 1 is a schematic diagram of an example of apparatus for multifrequency bioimpedance measurement;

FIG. 2 is a schematic diagram of the relationship between the Cartesian and Polar impedance notation;

FIG. 3 is a schematic diagram of an example of the processing system of FIG. 1;

FIG. 4 is a schematic diagram of a specific example of apparatus for impedance measurement; and,

FIG. 5 is a schematic diagram of a specific example of a signal generator for use in the apparatus of FIG. 1 or 4.

### DETAILED DESCRIPTION OF CERTAIN EMBODIMENTS

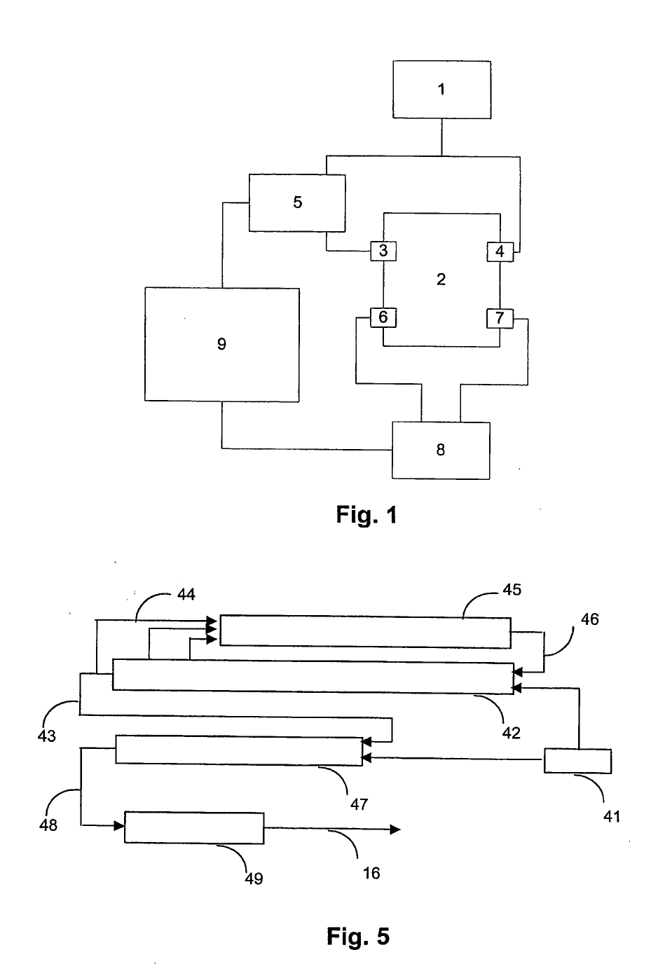

An embodiment of an apparatus suitable for measuring bioimpedance using multiple frequencies is shown in FIG. 1.

As shown the apparatus is formed from a signal generator 1, coupled to a body 2, such as a human subject, or the like, via electrodes 3, 4. A current detector 5 is coupled to the signal generator 1 and one of the electrodes 3, with a voltage detector 6 being coupled to the body 2 via respective electrodes 6, 7, as shown. Signals from the current and voltage detectors 5, 7 are transferred to a processing system 9 for subsequent processing.

In one embodiment, the signal generator operates to apply an electrical signal to the body 2, via the electrodes 3, 4. The current flow through the body is measured using the current detector 5, and transferred to the processing system 9. Simultaneously, the voltage generated across the body is measured using the voltage detector 6, and transferred to the processing system 9, thereby allowing the processing system 9 to determine the impedance of the body 2.

In particular, the impedance is calculated using the formula:
Z=V/I (1)
Where:
Z=impedance;
V=voltage; and,
I=current.

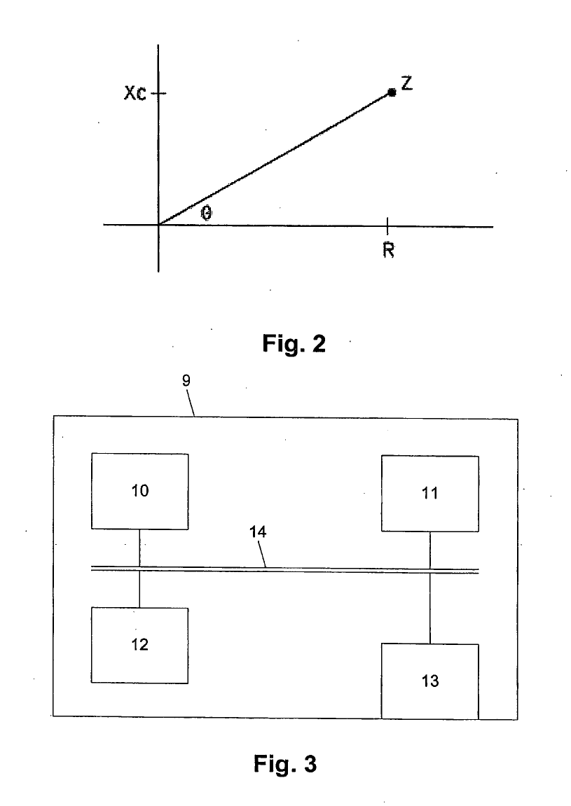

For complex impedance, each of these three values is represented by a complex vector. A complex vector can be represented in two ways, using either polar or Cartesian coordinates. Polar notation uses the vector's length (Z) and its phase (θ). The same information can also be described using Cartesian coordinates where the vector's X component is described as Resistance (R), and Y component is described as Reactance (Xc). This is shown for example in FIG. 2.

The impedance of the body 2 can be measured at one particular frequency f by applying a pure sine wave current having the frequency f to the body and measuring the applied current and the voltage developed across the body 2. The determined voltage and current can then be used to determine the impedance.

If the calculations are to be performed digitally, the current and voltage measurements need to be sampled at a measurement rate of at least 2×f, but realistically, to achieve good performance, a typical measurement rate should be higher, for example at 5×f. This is required to prevent problems with aliasing of the sampled signals. It will be appreciated that the more measurements taken, the more accurate the subsequent calculations will be. A typical number would be in the region of several thousand measurement points.

The apparatus described above operates to perform multiple frequency impedance measurements thereby allowing the system to determine the impedance for a number of different applied frequencies of signal f₁, f₂, . . . fₙ. In order to achieve this, the apparatus uses the principle of superimposition to allow the impedance calculations to be performed for multiple frequencies simultaneously.

In one example, this is achieved by having the signal generator generate an electrical signal formed from the summation of multiple sine waves. Accordingly, the signal generator operates to superimpose a number of sine waves and use these to form the electrical signal to be applied to the body 2. Ideally the resulting electrical signal should be formed from a superimposition of a number of waves, each having an equal amplitude.

The resulting current and voltages across the body 2 are then transferred to the processing system 9 to allow the processing system 9 to determine the impedance. Accordingly, it will be appreciated that any form of suitably adapted processing system may be used.

An embodiment of a suitable processing system is shown generally in FIG. 3. In particular, the processing system 9 includes a processor 10, a memory 11, an optional input/output (I/O) device 12, such as a keyboard and monitor, or the like, and an interface 13 coupled together via a bus 14. In use, the interface 13 is adapted to receive signals from the current and voltage detectors 5, 8. The processor 10 then executes applications software stored in the memory 11, to process the received signals.

Accordingly, it will be appreciated that embodiments of a processing system may be formed from any one of a number of forms of processing system, such as a suitably programmed PC, Lap-top, hand held PC, palm-top or the like. Alternatively, the processing system 9 may be formed from specialised hardware, such as an electronic touch sensitive screen coupled to suitable processor and memory.

In this embodiment, the processing system 9 operates to perform the impedance calculations by converting time-domain sequences of voltage and current measurements obtained from the current and voltage detectors 5, 8 into frequency-domain data. This is typically (and most efficiently) performed using a FFT. A single pure sine wave of frequency f in the time domain will appear as a thin single peak at frequency f in the frequency domain (frequency spectrum), with the height of the peak being proportional to the amplitude of the sine wave in the time domain. The FFT will also provide the phase (θ) of the sine wave, referenced to the start of the measurement period.

If the FFT operation is performed on both the voltage and current measurements, two peaks will result in the frequency spectrum, at the same frequency, but at differing heights corresponding to the amplitudes of the voltage and current sine waves. If these two heights are divided by each other, the impedance is determined (as given by the formula (1) above, Z=V/I).

If the phases are subtracted from each other, the phase of the impedance vector is determined. In this way, both values needed to define the impedance vector, namely its length (Z) and phase (θ), are determined. That is, the impedance vector is obtained by two simple FFT operations, one divide, and one subtraction.

As mentioned above, this embodiment allows the impedance to be calculated for multiple frequencies of interest simultaneously. Accordingly, the electrical signal applied to the body 2 is formed from a superimposition of multiple sine waves. Subsequently a FFT is performed on the measured current and the voltage, and a division and a subtraction is carried out for each point in the frequency spectrum. It will be appreciated that this process can be performed very rapidly, typically within a few milliseconds.

The more sine waves that are superimposed, the more points will be determined in the resulting frequency spectrum, and the more accurate the resulting plots. This principle can be maximised by applying a ‘white noise’ current to the body to be measured. An ideal white noise source contains equal amplitudes of all frequencies of interest. Accordingly, the use of an “ideal white noise” would allow the measurement of impedance at any number of frequencies simultaneously.

However, generating ideal white noise can be problematic, and accordingly, it is typical for the sample length of the white noise to be selected based on factors, such as the processing power available and the resolution required.

For example, if the white noise is selected to have a sample length of 1024 points, this will give 1024 separate points in the frequency domain, generating 1024 points on the resulting Cole-Cole plot.

However, in embodiments of the invention with sufficient processing power and storage memory, larger numbers of points can be used, giving a very high resolution. Thus, the larger the sample length, the higher the resolution of the resulting plot. However, it will be appreciated that the use of more sample points requires a corresponding increase in the processing power required to process the measured voltage and current signals. Thus, it is typical to select a sample length based on the implementation and the circumstances in which the invention is implemented, to thereby allowing the highest resolution to be determined based on the processing power available. This allows a wide range of

Furthermore, for a practical measurement of impedance, the white noise needs to be ‘band-limited’ where it will only contain frequency components up to a certain frequency f. The A/D conversion sample rate must be at least 2×f, to avoid “aliasing” errors in the processing, but realistically should be around at least 5×f for a practical device.

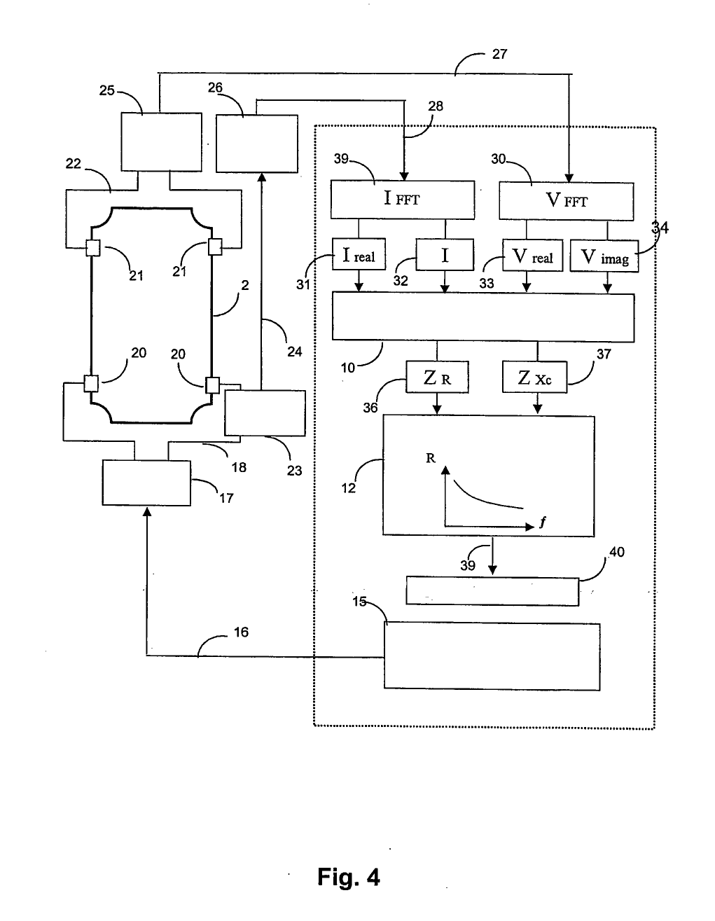

A specific embodiment of an apparatus suitable for performing impedance determination will now be described with reference to the FIG. 4.

In this embodiment, the apparatus uses specialised digital electronics to perform the functionality outlined above with respect to FIG. 1.

In particular, in this embodiment, the signal generator is formed from a pseudo-random voltage generator 15, coupled to a current source 17, which is in turn coupled to the body 2 via electrodes 20.

Two further electrodes 21 are coupled to an A/D converter 25 to form the voltage detector 8, with the current detector being formed from a current sampler 23 and an associated A/D converter 26.

The A/D converters 25, 26 are then coupled to processing electronics shown by the dotted lines, which may be implemented either as respective digital electronics, the processing system 9, or a combination of the two. In this embodiment, separate digital electronics and a processor 35 are used, as will be described below.

Operation of the system will now be described. In particular, the pseudo-random voltage generator 15, delivers an analog command voltage 16 to the current source 17. The current source 17 is responsive to the received command voltage 16 to generate a pseudo-random “white noise” current 18, which is comprised of multiple frequencies, and which is applied to the two electrodes 20.

The two electrodes 21 are used to measure the voltage 22 generated across the body 2, with the voltage 22 being digitized by the A/D converter 25. In addition to this, the current sampler 23 samples the pseudo-random current 18 and the resulting signal 24 is digitized by the A/D converter 26.

In this embodiment, the A/D converters 25, 26 will obtain measurements of the voltage 22 and the current signal 24 at a frequency that is at least five times greater than the maximum frequency of the applied current 18. It will therefore be appreciated that the sampling frequency will be selected based on the preferred implementation. Thus, for example, the sampling frequency may be between 4 MHz and 5 MHz, although any suitable frequency may be used depending on the circumstances. Furthermore, as mentioned above a range of sampling lengths may be used, although in one example, the sample lengths can be 1024 bits.

As mentioned above, it will be appreciated that the greater the sample length, and sample rate the more accurate the process will be. However, the use of larger a sample length and/or rate will lead to a corresponding increase in the amount of data processing that will be required. Accordingly, the use of 1024 bit samples, and a sampling rate of between 4 MHz and 5 MHz are illustrative only, but are particularly useful for providing good accuracy, without requiring undue processing. As processing systems and other digital electronics improve, it will be appreciated that higher sample lengths and rates will be achievable without effecting the time taken to obtain the readings.

The digital signal 27 resulting from the A/D conversion of the voltage 22 undergoes a FFT operation 30, which generates real and imaginary voltage components 33, 34 for multiple frequencies. Similarly the digital signal 28 output from the A/D conversion of the signal 24 undergoes a FFT operation 29, which generates real and imaginary components 31, 32 multiple frequencies.

It will be appreciated that the performance of the FFTs may be performed by the processor 10, or may alternatively be performed by separate processing electronics, as shown in this example. In any event, it will be appreciated that the signals received from the A/D converters 25, 26 may need to be temporarily stored, for example in either the memory 11, or separate memory such as a shift register or the like, before being processed.

These real and imaginary components 31, 32, 33 and 34 generated by the FFT are transferred to the processor 10, where the resistive and reactive components 36, 37 of the impedance for multiple frequencies are determined.

The resistive and reactive components 36, 37 can then be further processed and analysed in the processor 10 and a zero-infinity kHz parallel multiple frequency plot, also referred to as a Cole-Cole plot, can be generated before being displayed on the output device 12.

The resultant data shown generally at 39 can be transferred to the memory 11 for storage, or can be transferred to an external device 40, or the processor 10 for further processing and analysis. This includes the averaging of results, or the like, as will be described in more detail below.

In one embodiment, the components 15, and 29-38 may be accomplished using digital circuitry, or suitably programmed processing systems.

An embodiment of a circuit suitable for use as the pseudo-random voltage generator 15, used for the generation of band-limited white noise, will now be described with reference to FIG. 5.

In particular, the circuit includes a fixed frequency clock 41, a serial shift register 42, an XOR gate 45, a serial input-parallel output shift register 47, and a D/A converter 49.

In use, the fixed frequency clock 41 clocks the serial shift register 42. Several signals 44 from various points in the serial shift register 42 are fed into the XOR gate 45, the output 46 of which is fed back to the input of the serial shift register 42.

The output 43 from the serial shift register 42 is fed to the input of the serial input-parallel output shift register 47, which is also clocked from the clock 41. After the required number of bits appropriate for the correct operation of the D/A converter 49 have been shifted into the shift register 47 the parallel output 48 from the shift register 47 is sent to the D/A converter 49, which then generates the analog command voltage 16.

Accordingly, the above described circuit shows a Linear Feedback Shift Register (LFSR) circuit that produces a pseudo-random digital sequence that is fed into the D/A converter 49. However, it will be appreciated that this represents only one technique for generating white noise, and other techniques can be used.

In the above example however, the random signal is based on a sequence that forms the contents of the serial shift register 42. This sequence should be of such a length that it does not repeat until after many successive measurements, which may be achieved for example by providing a signal that is 100 bits in length, which will typically lead to a repeat time of several hundred million years.

This is important because in one embodiment, the apparatus described above is used to perform the measurement of the impedance over multiple frequencies a large number of times and then average the result.

In particular, it will be appreciated that in the idealised case, the signal applied to the body 2 has an equal amplitude for each applied frequency. Accordingly, the relative magnitudes obtained for impedance measurements at different frequencies will not be influenced by the applied signal.

However, if a random signal is used, it will be appreciated that the magnitude of the signal will vary from instant to instant. Accordingly, the impedance measured at any one time will depend to a degree on the magnitude of the applied signal at that time.

If the signal is truly random, then repeating impedance measurements a number of times and then averaging the results, will average out any variations in the resulting impedance that has arisen due to peaks and troughs in the applied signal.

However, in a pseudo random signal, it will be appreciated that any variations in the magnitude of the applied signal will repeat with some time period. Accordingly, if the sampling rate and repeat period happened to coincide, this may lead to exaggerated impedance measurements being obtained. By ensuring that the repeat time for the applied signal is significantly greater than the time periods over which measurements are taken, this problem is avoided.

Accordingly, it will be appreciated that the above described systems can be used to determine the impedance of a body at several frequencies simultaneously. This vastly reduces the length of time required to determine the impedance of an body, and in particular can reduce the time taken from several minutes achieved with existing techniques to a matter of milliseconds.

This in turn, allows repeat measurements to be performed over a short time period, such as a number of seconds, allowing the results from several readings to be averaged, thereby resulting in even more accurate results.

Another advantage of certain embodiments is that the circuit required to undertake the above operations can be almost entirely digital, giving the usual advantages of digital circuitry, namely, repeatability, reliability, no drift over either temperature or time, and simplicity of operation.

Persons skilled in the art will appreciate that numerous variations and modifications will become apparent. All such variations and modifications which become apparent to persons skilled in the art, should be considered to fall within the spirit and scope that the invention broadly appearing before described.

Thus, for example, it will be appreciated that the above described techniques may be utilised to determine the bioelectric impedance of a biological sample, and is not restricted to applications for humans, or the like.

What is claimed is:

1. A method of determining the impedance of a subject, comprising:
applying an electrical signal representing a range of superimposed frequencies;
determining, for a number of frequencies within the range, the current flow through the subject and the voltage across the subject; and
determining the impedance of the subject at each of the number of frequencies.

2. The method of claim 1, further comprising
generating component signals, each component signal having a respective one of the number of frequencies; and,
superpimosing the component signals to generate the electrical signal.

3. The method of claim 1, wherein the electrical signal is formed from white noise.

4. The method of claim 3, additionally comprising:
generating the white noise using a Linear Feedback Shift Register (LFSR) circuit to produce a pseudo-random digital sequence;
converting the pseudo-random digital sequence to an analog signal using a digital to analog (D/A) converter; and,
applying the analog signal to the subject.

5. The method of claim 1, wherein determining the current flow comprises:
sampling the current of the electrical signal applied to the subject; and
converting the current signal to a digitized current signal.

6. The method of claim 5, wherein determining the voltage comprises:
obtaining a signal representing the voltage generated across the subject; and
converting the voltage signal to a digitized voltage signal.

7. The method of claim 6, additionally comprising digitizing the current and voltage signals by sampling the signals at a predetermined sample rate.

8. The method of claim 6, additionally comprising digitizing the current and voltage signals by sampling the signals with a predetermined sample length.

9. The method of claim 6, additionally comprising converting each of the digitized voltage and current signals into the frequency domain.

10. The method of claim 9, wherein the conversion is performed using a Fast Fourier Transform (FFT).

11. The method of claim 9, further comprising:
receiving the converted voltage and current signals; and,
determining the impedance of the subject at each of the number of frequencies.

12. The method of claim 11, wherein the processing system is further adapted to determine the variation in the impedance with the frequency of the applied signal.

13. The method of claim 12, additionally comprising generating a graphical representation of the variation in the impedance with the frequency of the applied signal.

14. An apparatus for determining the impedance of a subject, comprising:
a signal generator configured to apply an electrical signal representing a range of superimposed frequencies;
a voltage detector configured to determine the voltage across the subject at a number of frequencies within the range;
a current detector configured to determine the current flow through the subject at a number of frequencies within the range; and,
a processing system configured to determine the impedance of the subject at each of the number of frequencies.

15. The apparatus of claim 14, wherein the signal generator is adapted to:
generate component signals, each component signal having a respective one of the number of frequencies; and,
superimpose the component signals to generate the electrical signal.

16. The apparatus of claim 14, wherein the electrical signal is formed from white noise.

17. The apparatus of claim 16, wherein the signal generator comprises:
a shift register circuit producing a pseudo-random digital sequence; and
a D/A converter converting the pseudo-random digital sequence to an analog signal.

18. The apparatus of claim 17, wherein the signal generator additionally comprises:
a shift register having an output coupled to the D/A converter; and
an exclusive OR (XOR) gate adapted to receive inputs from a number of predetermined locations in the first register, logically combine the inputs to generate an XOR output, and provide the XOR output to an input of the shift register;

19. The apparatus of claim 18, wherein the signal generator additionally comprises a second shift register, the second shift register being adapted to couple an output of the first shift register to an input of the D/A converter.

20. The apparatus of claim 14, wherein the current detector comprises:
a current sampler coupled to the signal generator for sampling the current flowing through the subject; and
a current analog to digital (A/D) converter for converting the sampled current to a digitized current signal.

21. The apparatus of claim 20, wherein the voltage detector comprises a voltage A/D converter coupled to the subject via a respective set of electrodes, the voltage A/D converter being adapted to generate a digitized voltage signal.

22. The apparatus of claim 21, wherein the current and voltage A/D converters are adapted to digitize the current and voltage signals by sampling the signals at a predetermined sample rate.

23. The apparatus of claim 21, wherein the current and voltage D/A converters are adapted to digitize the current and voltage signals by sampling the signals with a predetermined sample length.

24. The apparatus of claim 21, wherein the processing system is adapted to convert each of the digitized voltage and current signals into the frequency domain.

25. The apparatus of claim 24, wherein the conversion is performed using a FFT.

26. The apparatus of claim 24, wherein the processing system comprises processing electronics for performing the conversion.

27. The apparatus of claim 24, wherein the processing system is adapted to:
receive the converted voltage and current signals; and
determine the impedance of the subject at each of the number of frequencies.

28. The apparatus of claim 14, wherein the processing system is further adapted to determine the variation in the impedance with the frequency of the applied signal.

29. The apparatus of claim 14, wherein the processing system is further adapted to generate a graphical representation of the variation in the impedance with the frequency of the applied signal.

30. A processing system for use in an apparatus for determining the impedance of a subject, wherein the processing system is adapted to:
receive a digitized current signal representing the current flow through the subject at a number of frequencies, for an applied electrical signal representing a range of superimposed frequencies;
receive a digitized voltage signal representing the voltage across the subject at a number of frequencies within the range;
convert each digitized signals into the frequency domain; and
determine the impedance of the subject at each of the number of frequencies.

31. The processing system of claim 30, wherein the conversion is performed using a FFT.

32. The processing system of claim 30, wherein the processing system includes processing electronics for performing the conversion.

33. The processing system of claim 30, wherein the processing system includes a processor for determining the impedance.

34. The processing system of claim 33, wherein the processor is further adapted to determine the variation in the impedance with the frequency of the applied signal.

35. The processing system of claim 34, wherein the processing system includes a display, and wherein the processor is adapted to generating a graphical representation of the variation in the impedance with the frequency of the applied signal.

36. An apparatus for determining the impedance of a subject, comprising:
means for applying an electrical signal representing a range of superimposed frequencies;
means for determining the current flow through the subject for a number of frequencies within the range;
means for determining the voltage across the subject for the number of frequencies; and
means for depermining the impedance of the subject at each of the number of frequencies.

37. The apparatus of claim 36, wherein the means for applying an electrical signal representing a range of superimposed frequencies comprise a means for generating an electrical signal formed from the summation of a plurality of sine waves.
